(19) United States
(12) Patent Application Publication
(10) Pub. No.: US 2007/0010750 A1
(43) Pub. Date: Jan. 11, 2007

(54) Biometric sensor and biometric method

(76) Inventors: Akinori Ueno, Tokyo (JP); Yoji Ishiyama, Tokyo (JP); Hiroshi Hoshino, Saitama (JP); Kenzou Kassai, Osaka (JP); Sachiyo Suzuki, Osaka (JP)

Correspondence Address: DITTHAVONG & MORI, P.C., 10507 BRADDOCK ROAD, SUITE A, FAIRFAX, VA, 22032, US

(21) Appl. No.: 10/574,558

(22) PCT Filed: Sep. 1, 2004
(86) PCT No.: PCT/JP04/12632

§ 371 Date: Apr. 3, 2006

(30) Foreign Application Priority Data
Oct. 3, 2003 (JP) 2003-346299

(51) Int. Cl. A61B 5/04

(52) U.S. Cl. 600/509; 600/388

(57) ABSTRACT

A living body measuring sensor (1) is made to contact a body surface of a measuring subject through capacitance coupling using a cloth (6) between a metal electrode (2) and the body surface as the capacitance, a living body electric signal is extracted from the metal electrode (2), and an elctrocardiographic waveform is outputted based on an output of the living body measuring sensor (1) using an impedance converter having a high input impedance and a low output impedance.

### TECHNICAL FIELD

The present invention relates to a living body measuring sensor and a living body measuring method, more particularly to a living body measuring sensor and a living body measuring method for obtaining an electrocardiogram without making a direct contact with a body surface of a measuring subject.

### BACKGROUND ART

An electrocardiogram obtained by a conventional electrocardiograph records a ventricular function measured at rest, wherein a variation of a voltage generated on a body surface of a measuring subject is recorded. The electrocardiogram is a record of an electric activity generated in a heart pulsation, wherein the voltage generated on the body surface as a result of a cardiac muscle excited by generation and propagation of stimulations prior to a cardiac contraction is recorded using a curved line.

FIG. 9 is a schematic block diagram of a conventional electrocardiograph. For the purpose of the measurement of the electrocardiogram, a fixed electrode 51 such as a silver/silver chloride electrode shown in FIG. 9 is made to closely contact a skin 10 using a conductive paste in around a wrist or an ankle of a measuring subject, adsorbed to the skin 10 by reducing a pressure or pressurized by a belt to be thereby fixed thereto. A living body electric signal obtained from the fixed electrode 51 is amplified by a differential amplifier 52, and a noise component thereof is eliminated by a noise eliminating filter 53, and further, the living body electric signal is sampled by an A/D converter 54 to be thereby converted into a digital signal. The digital signal is then processed by a processing device 55 so that an electrocardiogram shown in FIG. 10A is recorded on a recorder or displayed in a waveform on a display screen.

In the conventional measurement, it is necessary for the measuring subject to lie on a medical examination table on his/her back and stay at rest. A fixed electrode 51 is fixed to the measuring subject in each measurement and formed from the conductive paste as described above. Further, the electrode is depressurized/pressurized and thereby fixed to the body surface prior to the commencement of the measurement. Therefore; there is a limit in conducting the measurement in such a manner that the measuring subject can be relaxed during the measurement.

Further, in the case of a patient having a paroxysmal or temporary heart disease, it is necessary to record the electrocardiogram by an electrocardiogram recorder, for example, for as long as 24 hours. The patient is, as in this case, forcibly left in the state where the fixed electrode 51 is attached to the body surface. When the fixed electrode 51 is attached to the body surface for as long as a few hours, the surface in contact with the electrode becomes itchy or an allergic reaction may cause the surface to be sore due to inflammation. If a cloth or the like is interposed between the fixed electrode 51 and a skin 10 so as to prevent the metal from directly contacting the skin 10, a living body electric signal cannot be directly detected from the fixed electrode 51.

A possible method is to detect the living body electric signal by capacitance-coupling the fixed electrode 51 via the cloth and thereby mounting the electrode on the skin 10. However, the method is rather awkward because an output of the fixed voltage 51 shows a high impedance and a noise voltage is thereby increased as shown in FIGS. 10B and 10C in response to an even slight amount of noise current, which results in a failure to retrieve the living body electric signal. FIG. 10B shows an output voltage of the fixed electrode 51 when silk is interposed, while FIG. 10C shows an output voltage thereof when cotton is interposed.

Japanese Unexamined Patent Application No. 2002-159458 recites a living body electric signal induction sensor and a recording system in which a conductive fiber is woven into a predetermined portion of a clothing, the living body electric signal is detected by the conductive fiber constituting an induction-electrode, and the electrocardiogram is recorded on a recorder housed in a pocket of the clothing.

However, in the case of using the conductive fiber as the induction electrode, the conductive fiber may fail to closely contact the skin, which cannot assure an accurate electrocardiogram. Further, the conductive fiber includes the risk of inducing the allergic reaction in the same manner as in the metal electrode.

### DISCLOSURE OF THE INVENTION

Therefore, an object of the present invention is to provide a living body measuring sensor and a living body measuring method capable of measuring an electrocardiogram using a capacitance in a less invasive manner.

The present invention relates to the living body measuring sensor for detecting a living body electric signal from a body surface of a measuring subject, which comprises a conductive electrode capacitance-coupled on the body surface of the measuring subject via an insulating member and a living body electric signal extractor circuit for extracting the living body electric signal from the conductive electrode as a low impedance signal.

According to the present invention, the conductive electrode is mounted on the body surface of the measuring subject via the insulating member and the living body electric signal is outputted as the low impedance signal. Thereby, the electrocardiogram can be measured in the less invasive manner without any adverse effect from the noise, and a risk of inducing an allergic reaction can be eliminated.

The conductive electrode is preferably a metal electrode.

The conductive electrode is preferably a conductive fiber.

The insulating member is preferably a thin cloth.

The living body electric signal extractor circuit preferably includes an impedance converter circuit whose input is a high input impedance and output is a low impedance.

The living body electric signal extractor circuit preferably further includes a filter circuit for extracting a frequency component including the living body electric signal from the output of the impedance converter circuit.

The living body electric signal extractor circuit preferably further includes an amplifier circuit for amplifying the living body electric signal outputted from the impedance converter circuit using a high gain.

A barium titanate porcelain may be provided as a high permittivity member to be provided between the conductive electrode and the insulating member.

The living body measuring method according to the present invention extracts the living body electric signal with the low impedance by capacitance-coupling and thereby mounting the living body measuring sensor including the conductive electrode on the body surface of the measuring subject via the insulating member and thereby mounting the sensor on the body surface of the measuring subject.

### BRIEF DESCRIPTION OF DRAWINGS

FIG. 1 is a sectional view of a living body measuring sensor according to an embodiment of the present invention.

FIG. 2 is a graph showing a relationship between a thickness of a cloth and a capacitance.

FIG. 3 is a graph showing a relationship between a frequency and an impedance.

FIG. 4 is a block diagram of a living body measuring device according to an embodiment of the present invention.

FIG. 5A is a graph showing an electrocardiographic waveform outputted from the living body measuring device shown in FIG. 4.

FIG. 5B is a graph showing an electrocardiographic waveform outputted from the living body measuring device shown in FIG. 4.

FIG. 6 is a sectional view of a living body measuring sensor according to another embodiment of the present invention.

FIG. 7A is a view showing a clothing for living body measurement constituting the living body measuring sensor according to still another embodiment of the present invention.

FIG. 7B is a view showing a clothing for living body measurement constituting the living body measuring sensor according to still another embodiment of the present invention.

FIG. 8A is an enlarged view of a conductive fiber of the clothing for the living body measurement shown in FIGS. 7A and 7B.

FIG. 8B is an enlarged view of a conductive fiber of the clothing for the living body measurement shown in FIGS. 7A and 7B.

FIG. 9 is a schematic block diagram of a conventional electrocardiograph.

FIG. 10A is a graph showing an electrocardiographic waveform outputted from the conventional electrocardiograph.

FIG. 10B is a graph showing an electrocardiographic waveform outputted from the conventional electrocardiograph.

FIG. 10C is a graph showing an electrocardiographic waveform outputted from the conventional electrocardiograph.

### BEST MODE FOR CARRYING OUT THE INVENTION

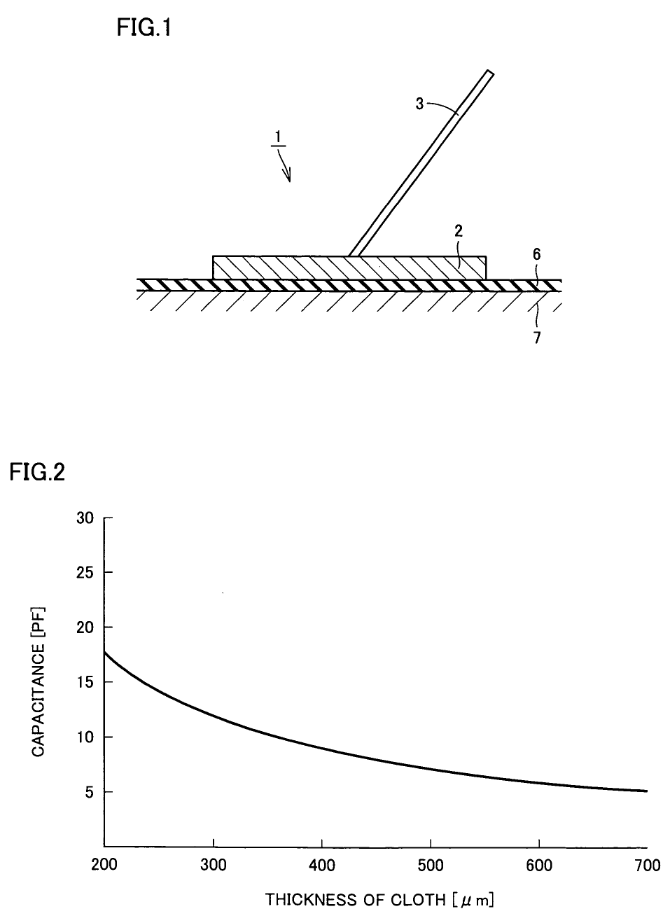

FIG. 1 is a sectional view of a living body measuring sensor according to an embodiment of the present invention. A living body measuring sensor 1 shown in FIG. 1 employs a contact made by means of capacitance coupling without any direct contact with a skin 7 of a measuring subject as a measurement principle. A silver electrode 2, which is an example of a metal electrode as a conductive electrode, is provided. The silver electrode 2 is formed in a thin disk shape or rectangular shape. The conductive electrode is not limited to the silver electrode 2, and may employ stainless, aluminum, a conductive cloth, a conductive gel or the like.

The living body measuring sensor 1 is brought into close contact with a surface of the skin 7 via a thin cloth 6 formed from silk or the like serving as an insulating member so as to detect a variation of a living body electric signal generated on the body surface of the measuring subject.

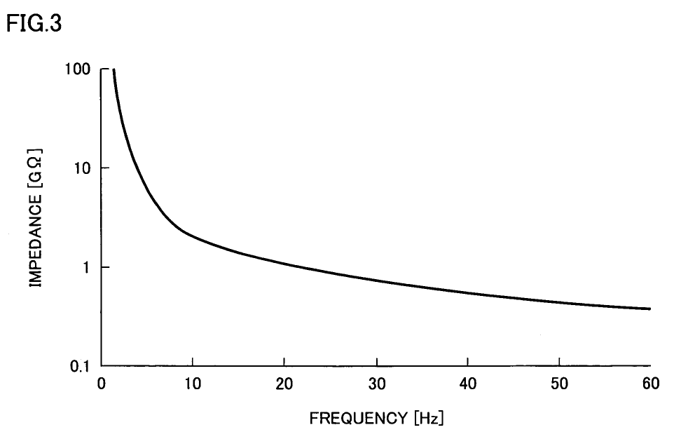

FIG. 2 is a graph showing a relationship between a thickness of the cloth and the capacitance. FIG. 3 is a graph showing a relationship between a frequency and an impedance.

As shown in FIG. 2, the capacitance increases as the thickness of the cloth is thinner. For example, when a silk cloth having a thickness of approximately 240 μm is used as the cloth 6, the capacitance between the living body measuring sensor 1 and the skin 7 is estimated to be approximately 10⁻¹¹ F. Further, it is learnt from FIG. 3 that an output impedance is lessened as a frequency f of a living-body waveform increases. Accordingly, it is estimated that an output impedance Z of the living body measuring sensor 1 in the state of interposing the silk results in a high impedance of approximately 10¹¹Ω at the frequency of 0.1 Hz.

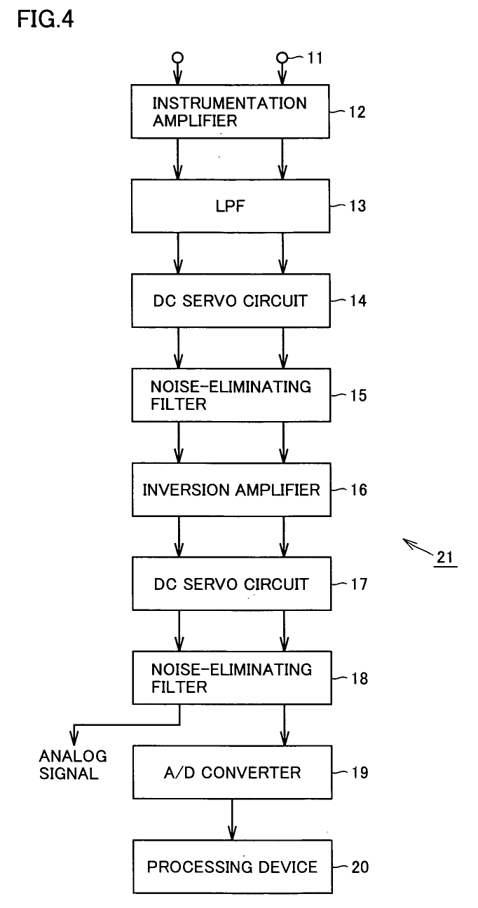

FIG. 4 is a block diagram of a living body measuring device 21 for outputting an electrocardiogram based on a living body electric signal outputted from the living body measuring sensor 1 shown in FIG. 1. As described above, the output impedance Z of the living body measuring sensor 1 shows such a high value as 10¹¹Ω, which results in the generation of a large noise voltage when even a slight amount of noise current is applied to the output. In order to deal with the disadvantage, an impedance converter for outputting the output signal of the living body measuring sensor 1 with a low impedance is necessary.

The living body electric signal of the high impedance detected by the living body measuring sensor 1 is supplied to an instrumentation amplifier 12 via an input terminal 11, and converted into the living body electric signal of the low impedance, and then supplied to an LPF (low-pass filter) 13. In the instrumentation amplifier 12, an input impedance is set to 1000 GΩ, and a gain is set to 62 times as a result of changing a value of an externally added resistance. The LPF 13 extracts a frequency component equal to or below 100 Hz from the living body electric signal and supplies a result of the extraction to a DC servo circuit 14. The DC servo circuit 14 applies the servo so that a variation of a DC component of the living body electric signal is controlled to be zero, which is supplied to a noise eliminating filter 15. The noise eliminating filter 15 is adapted to be switched upon necessity so that the frequency component of 50 Hz or 60 Hz can be extracted from the living body electric signal, and supplies the living body electric signal of the extracted frequency component to an inversion amplifier 16.

The inversion amplifier 16 amplifies the living body electric signal, which is inverted by the instrumentation amplifier 12, by 16 times, and inverts the living body electric signal to an initial polarity of the signal. Accordingly, the living body electric signal is amplified by 62×16≅1000 times. The inverted living body electric signal is supplied to a DC servo circuit 17, which applies the servo in the same manner as the DC servo circuit 14 so that the variation of the DC component of the living body electric signal becomes zero, and supplies it to a noise eliminating filter 18. The noise eliminating filter 18 is adapted to be switched upon necessity so that the frequency component of 50 Hz or 60 Hz can be extracted from the living body electric signal in the same manner as the noise eliminating filter 15 in the previous stage. The living body electric signal extracted by the noise eliminating filter 18 is sampled by an A/D converter 19 and converted into a digital signal, and then supplied to a processing device 20 to be subjected to necessary processes. As a result, the electrocardiographic waveform is outputted.

As another possible constitution, an analog living body electric signal is outputted from the noise eliminating filter 18 and the electrocardiographic waveform is observed by an oscilloscope.

FIGS. 5A and 5B are graphs of electrocardiographic waveforms outputted from the living body measuring device shown in FIG. 4, respectively showing the electrocardiographic waveform outputted when silk is interposed between the living body measuring sensor 1 and the skin 7, and the electrocardiographic waveform outputted when cotton is interposed therebetween.

As described above, according to the present embodiment, the silver electrode 2 of the living body measuring sensor 1 is brought into close contact with the skin 7 of the measuring subject via the cloth 6, the device whose input impedance is set to be higher is used as the instrumentation amplifier 1 of the living body measuring device 21, the DC servo circuits 14 and 17 provided in two stages apply the servo so as to lead the variation of the DC component to be zero, and the noise eliminating filters 15 and 18 provided in two stages select and extract one of the frequency bands of 50 Hz and 60 Hz from the living body electric signal. Thereby, the electrocardiographic waveform can be outputted.

Therefore, the electrocardiogram can be measured in a less invasive manner by mounting the living body measuring sensor 1 on an underwear formed from silk, cotton or the like. Further, a risk of inducing an allergic reaction, which was generated by the conventional method of directly mounting the fixed electrode mounted on the body, can be eliminated because the living body measuring sensor 1 is mounted on the skin via the underwear or the like.

The cloth interposed between the living body measuring sensor 1 and the body surface of the measuring subject is not limited to silk or cotton, and may employ a synthetic fiber or Japanese paper having a thickness approximately equal to that of the cloth formed from any of the foregoing materials.

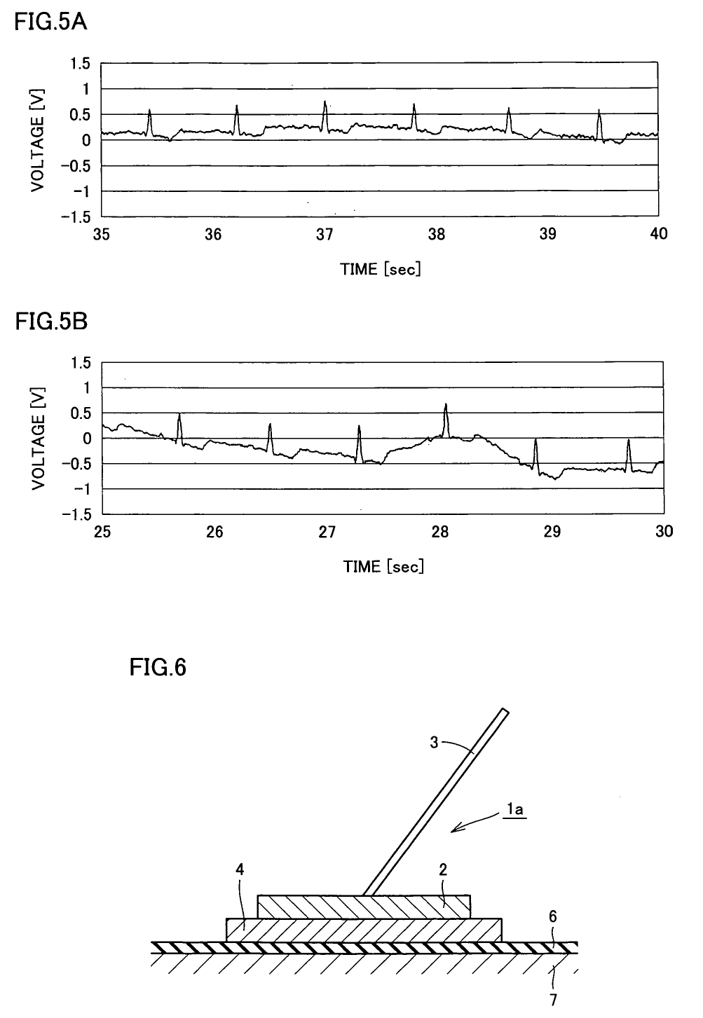

FIG. 6 is a sectional view of a living body measuring sensor according to another embodiment of the present invention. A living body measuring sensor 1a shown in FIG. 6 is further provided with a barium titanate (BaTiO₃) porcelain 4 as a high permittivity material between the metal electrode 2 of the living body measuring sensor 1 and the cloth 6 shown in FIG. 1. The barium titanate porcelain 4 is formed in a disk shape or rectangular shape, and one surface of the silver electrode 2 is in close contact with and electrically connected to one surface of the barium titanate porcelain 4. Because the capacitance can be increased by thus providing the barium titanate porcelain 4 with respect to the living body measuring sensor 1a, the output impedance of the sensor can be lessened in comparison to the embodiment shown in FIG. 1, and the input impedance of the measuring device can be lessened in comparison to the example shown in FIG. 4. Therefore, the impedance converter circuit whose input impedance of approximately 100 MΩ can be used.

As described above, according to the present embodiment, the living body measuring sensor 1a comprising the silver electrode 2 making a close contact with the one surface of the barium titanate porcelain 4 and retrieving the living body electric signal is disposed on the skin 7 of the measuring subject via the thin cloth 6, the barium titanate porcelain 4 and the thin cloth 6 are capacitance-coupled, the living body electric signal is retrieved from the silver electrode 2, and the output of the living body measuring sensor 1 is supplied to the living body measuring device so as to output the electrocardiogram.

In the foregoing description, the application of the barium titanate porcelain 4 as the high permittivity member was described. However, the present invention is not limited to the material, and may employ a high permittivity member of some other type.

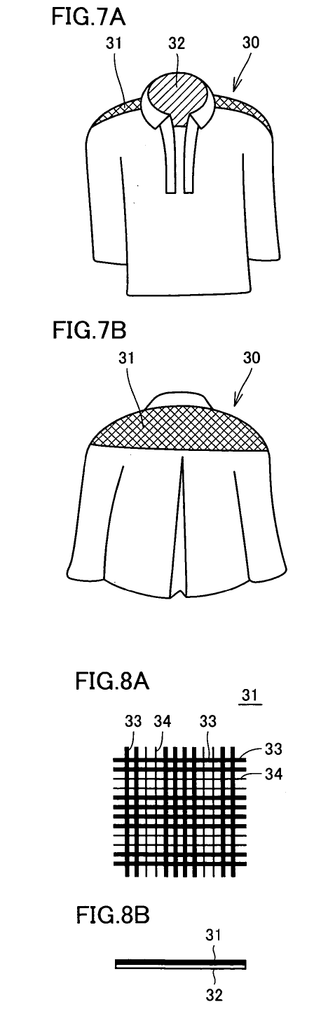

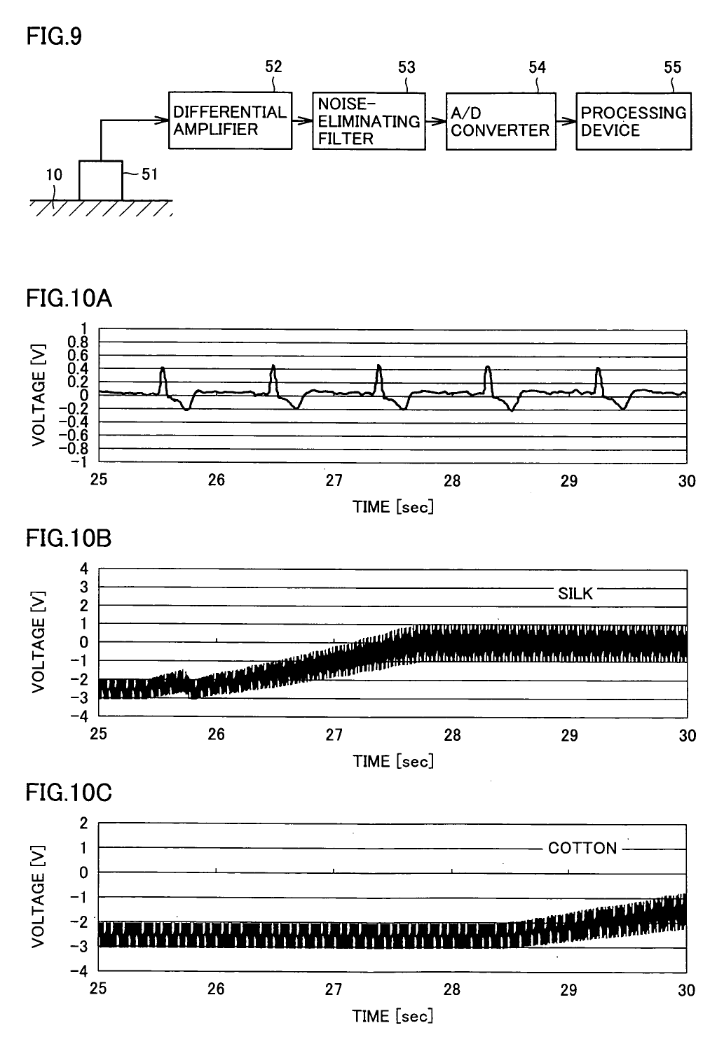

FIGS. 7A and 7B each shows a clothing for living body measurement constituting a living body measuring sensor according to still another embodiment of the present invention. FIGS. 8A and 8B are enlarged views of a conductive fiber of the clothing for the living body measurement shown in FIGS. 7A and 7B.

The living body measuring sensors 1 and 1a respectively shown in FIGS. 1 and 6 are adapted to closely contact the skin 7 via the cloth such as the underwear. In the embodiment shown in FIGS. 7A and 7B, a conductive fabric 31 is incorporated into a shoulder portion of a clothing 30 which is a position constantly in direct contact with the body surface of the measuring subject. A silk 32 is incorporated into between the conductive fabric 31 and the body surface so that the conductive fabric 31 does not directly contact the body surface of the measuring subject.

A woven body formed from a conductive yarn 33 and a non-conductive yarn 34 constitutes the conductive fabric 31 as shown in FIG. 8A, and the silk 32 shown in FIG. 8B is incorporated into between the woven body and the body surface. The conductive yarn 33 can employ, for example, a metal yarn such as gold, silver or copper, a conductive polymer such as polyaniline or polyacetylene or a conductive fiber such as a silver-plated nylon yarn. The non-conductive yarn 34 can employ a cotton yarn, acryl, nylon, a polyester yarn or the like.

Connecting the conductive fabric 31 to the input terminal 11 of the living body measuring device 21 shown in FIG. 4, the electrocardiographic waveform can be outputted from the processing device 20.

In the embodiment shown in FIGS. 7A and 7B, the conductive fabric 31 is incorporated into the shoulder portion of the clothing 30. However, the present invention is not limited to the constitution as far as the conductive fabric 31 is incorporated into a position capable of constantly making a direct contact with the body surface of the measuring subject. The conductive fabric 31 may constitute the entire clothing 30.

Thus far, the preferred embodiments of the present invention were described referring to the drawings, however, the present invention is not limited to the embodiments shown in the figures. The shown embodiments can be modified and corrected in various manners within the scope identical to the present invention and the scope of its equivalence.

### INDUSTRIAL APPLICABILITY

The present invention, wherein the living body measuring sensor 1 is brought into contact with a body surface of a measuring subject by means of the capacitance coupling using the cloth 6 between the metal electrode 2 and the body surface of the measuring subject as the capacitance, the living body electric signal is extracted from the metal electrode 2, and the output of the living body measuring sensor 1 is supplied to the living body measuring device 21 including the impedance converter having the high input impedance and low output impedance so as to read the voltage waveform, can be utilized in measuring the electrocardiogram in the less a invasive manner.

1-10. (canceled)

11. A living body measuring sensor for detecting a living body electric signal from a body surface of a measuring subject, comprising
a conductive electrode capacitance-coupled on said body surface of said measuring subject via an insulating member; and
a living body electric signal extractor circuit for outputting said living body electric signal from said conductive electrode with a low impedance.

12. The living body measuring sensor as claimed in claim 11, wherein said conductive electrode is a metal electrode.

13. The living body measuring sensor as claimed in claim 11, wherein said conductive electrode is a conductive fiber.

14. The living body measuring sensor as claimed in claim 11, wherein said insulating member is a thin cloth.

15. The living body measuring sensor as claimed in claim 11, wherein said living body electric signal extractor circuit includes an impedance converter circuit whose input is a high input impedance and output is a low impedance.

16. The living body measuring sensor as claimed in claim 15, wherein said living body electric signal extractor circuit includes a filter circuit for extracting a frequency component including said living body electric signal from an output of said impedance converter circuit.

17. The living body measuring sensor as claimed in claim 15, wherein said living body electric signal extractor circuit includes an amplifier circuit for amplifying said living body electric signal outputted from said impedance converter circuit using a high gain.

18. The living body measuring sensor as claimed in claim 11, further
including a high permittivity member to be provided between said conductive electrode and said insulating member.

19. The living body measuring sensor as claimed in claim 18, wherein said high permittivity member is a barium titanate porcelain.

20. A living body measuring method for extracting a living body electric signal from a body surface of a measuring subject using a living body measuring sensor including a conductive electrode mounted on said body surface of said measuring subject via an insulating material, wherein
said living body electric signal is outputted with a low impedance by capacitance coupling and thereby mounting said living body measuring sensor on said body surface of said measuring subject.
